(19) United States
(12) Patent Application Publication
(10) Pub. No.: US 2007/0275067 A1
(43) Pub. Date: Nov. 29, 2007

(54) Compression Coated Tablet Comprising Sumatriptan

(75) Inventors: Hiteshkumar Doshi, Mumbai (IN); Parizad Elchidana, Mumbai (IN); Sunil Jog, Mumbai (IN); Deepak Sonaje, Mumbai (IN)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Norton Healthcare Ltd., London (GB)

(21) Appl. No.: 10/585,547

(22) PCT Filed: Jan. 8, 2005
(86) PCT No.: PCT/US05/00500

§ 371 Date: May 23, 2007

(30) Foreign Application Priority Data
Jan. 9, 2004 (GB) 0400452.9

(51) Int. Cl. A61K 31/404; A61K 9/28; A61P 25/06

(52) U.S. Cl. 424/474; 514/415

(57) ABSTRACT

The present invention provides a pharmaceutical composition for oral administration, comprising a core of active ingredient and an outer non-active layer formed on the core by application of pressure.

### BACKGROUND OF THE INVENTION

1. Field of the Invention

The present invention relates to pharmaceutical compositions for oral administration of prophylactic and therapeutic active materials or combinations thereof and methods of making the same. The invention claimed herein is for compression coated tablets that improve the taste and palatability of tablets containing unpleasant tasting active ingredients.

Tablet compositions offer many advantages, including ease of product handling, chemical and physical stability, portability (in particular, allowing ready availability to the consumer when needed), aesthetic acceptability and dosage precision, i.e., ensuring consistent and accurate dosages of the pharmaceutical active.

One important factor in formulating tablets is palatability and mouth feet especially in tablets that include pharmaceutical dosages. However, many pharmaceutical ingredients have both an unpleasant mouth feel and unpalatable taste due to bitterness, chalkiness, grittiness, dryness and astringent properties of these materials. Accordingly, the practical value of these materials is substantially diminished due to poor patient compliance.

Active agents such as water soluble drug materials like sumatriptan and its salt or solvate, cetirizine, metronidazole, quinine and its salts etc generally have an unpleasant and bitter taste. When drug materials like sumatriptan and its salt or solvate are administered orally their unpleasant taste may exacerbate nausea and vomiting associated with migraine. In order to circumvent this limitation, it would be useful to improve the palatability of sumatriptan succinate.

A number of formulations have accordingly been investigated to improve the mouth feel and palatability of such compositions.

Frisbee et al, U.S. Pat. No. 6,013,280 discloses a self binding, glycerin free tablettable pharmaceutical composition comprising saccharide carriers, sugar alcohols such as Sorbitol and xylitol and therapeutic agent for example Sumatriptan succinate.

Frisbee et al, U.S. Pat. No. 6,086,920 discloses a pharmaceutical dosage form containing micro spheres which may have the taste masked and which disintegrates quickly in water. The micro spheres are composed of a therapeutic agent, for example Sumatriptan succinate, disintegrate(s) and spheronisation aids.

Ahlgren et al, U.S. Pat. No. 6,117,452 discloses preparation of thermoformed particulates of active agents for example Sumatriptan succinate via processes, which employ certain combinations of fatty esters and optional surfactants or emulsifiers as processing aids. Micro spheres are preferred particulates because they can be readily treated with taste masking and control release coatings. Micro spheres are made by a spheronisation process.

Mezaache et al, U.S. Pat. No. 6,165,512 discloses an oral solid dosage form such as tablets and lozenges which when ingested quickly dissolve in the mouth but which effectively mask the taste of an unpleasant therapeutic agent for example sumatriptan succinate therein. The disclosure relates to shapeable compositions to be used to make an oral dosage form containing coated liquidflash particles which contain therapeutic agent for example sumatriptan succinate, solubilizer and spheronisation aids. Blending of these coated particles with glycerin free bodies and shaping the blend produces the dosage form.

Phillips et al, U.S. Pat. No. 6,368,627 discloses a pharmaceutical composition for oral administration which comprises a film coated solid dosage form including Sumatriptan succinate as active ingredient. This method is time consuming and expensive to produce as the core tablet needs to be film coated to mask the bitter taste of Sumatriptan succinate.

Robinson et at U.S. Pat. No. 6,488,961 discloses effervescent granules having a controlled rate of effervescence. Such granules comprise an acidic agent, an alkalinizing agent hot melt extrudable binder and therapeutic agent such as Sumatriptan succinate.

Cherukuri et a, U.S. Pat. No. 6,589,556 discloses a rapid melt semisolid molded composition of therapeutic agent for example Sumatriptan succinate for better taste and mouth feel. The rapid melt semisolid molded composition contains at least one binder, salivating agent, therapeutic agent and bulking agent.

These prior art compositions have various disadvantages. For example, the respective methods of preparation are time consuming and expensive. We have found that some of these methods do not suitably improve the taste and mouth feel of the resulting products.

### SUMMARY OF THE INVENTION

According to the invention there is provided a pharmaceutical composition for oral administration, comprising a core of active ingredient and an outer non-active layer formed on the core by application of pressure.

Using the invention it is possible to provide a pharmaceutical composition for oral administration which affords a better taste, and storage stability, than those known from the prior art.

There may be a plurality of superposed non-active layer parts forming the outer layer. This provides a way of building up a cohesive outer layer, or coat. Each layer forming the outer layer may be applied to the core by a compression method to form the outer layer.

Each layer from a layer adjacent the core to an outermost layer may be formed in turn by application of pressure to form the outer layer. This assists in generating a cohesive outer coat.

The outer non-active layer may comprise granules which are compressed onto the core to form said outer layer. This again goes to providing a cohesive outer layer or coat.

The granules may suitably be formed by a wet or dry granulation, or by direct blending process.

Important physical-mechanical characteristics include the size, shape, compressibility, moisture content, and lubrication properties of the materials. Also important is the type of the material being compressed e.g. whether it is a powder or granule and the relative proportions of active agents, diluents and lubricating agents.

Tablets may be manufactured by wet granulation, dry granulation, compaction, or direct compression or other methods known to the person skilled in the art.

Compressed tablets may have coating on the outer layers. Types of coating include sugar coating, film coating, or a functional coating that allows delayed or controlled release of the active agent Such coatings being known to the person skilled in the art.

The outer layer may encase substantially the whole surface area of the core. This provides for a cohesive, integrated coat integral with the core.

An outer surface of the outer layer may comprise a surface profile. This provides for marking the composition with markings required by regulatory authorities.

The surface profile of a tablet depends upon several factors, for example, the physical-mechanical properties of the active agent and coating materials and the processes used to compress and/or coat the active agent material.

The surface profile may comprise an embossed or debossed surface profile, or an engraved surface profile, or an intaglio surface profile.

Debossing, engraving, indicia, and embossing for tablet identification are formed during tablet formation Modifications in tooling for the outer layer of tablet help in formation of tablets with debossed, engraved, indicia, or embossed surface profile. Debossing is the preferred choice and allows ease in packaging and handling. However debossing affects the surface profile of a tablet when a specialized functional coating is used. It also detrimentally affects taste masking and the effectiveness of functional coatings such as enteric coatings and extended release coatings.

The present invention allows the use of debossing for tablet identification without the associated detrimental affect to the masking function of the outer coating layer. The effectiveness of functional or film coatings is also maintained.

The term “intaglio” refers to a printing technique in which the image is engraved into a surface e.g. the surface of a tablet The engraving is then inked and rubbed clean so that the only ink remaining is that present in the engraving. Intaglio printing is frequently used as an anti-counterfeiting measure. Traditionally copper or zinc plates are used, and the incisions are created by etching or engraving the image or using mezzotint. In printing, the surface is covered in ink, and then rubbed vigorously with tarlatan cloth or newspaper to remove the ink from the surface, leaving it in the incisions. A damp piece of paper is placed on top, and the plate and paper are run through a printing press that, through pressure, transfers the ink to the paper. This method has been adapted by persons skilled in the art for the printing of tablet identification onto tablets.

The use of intaglio as an overprinting process for identification of tablets take place after tablet formation and/or coating of the tablet The process typically requires printing ink, vehicle for, and specialized equipment and may involve the use of organic solvents.

The term “tablet identification” means the application of any logo, product name or company name, identification code, or character to a tablet by means of debossing or embossing or other means known to be suitable by a person skilled in the art.

The composition may also comprise an applied indicia.

The term “indicia” means any discriminating mark, sign, token, indication or appearance.

The applied indicia may comprise a printed indicia. This may be essential for regulatory marking and R.T.M. notice.

The outer non-active layer may comprise one or more pharmaceutical carriers or excipients.

Suitably, the outer layer may comprise lubricating agent such as magnesium stearate 0.1 to 5%, filler such as lactose 30 to 90% and microcrystalline cellulose 5 to 30%, and disintegrating agent such as croscarmellose sodium 0.05 to 15%.

The core may also comprise one or more phanmaceutical carriers or excipients.

The core may comprise one or more active ingredients in the range 0.01 mg to 100 mg.

Additionally, the outer layer may be film coated for aesthetic or functional purposes.

Other improvement which the present invention provides over the prior art will be identified as a result of the following description which sets forth the preferred embodiments of the present invention. The description is not in any way intended to limit the scope of the present invention, but rather only to provide a working example of the presently preferred embodiments with reference to the accompanying drawings in which.

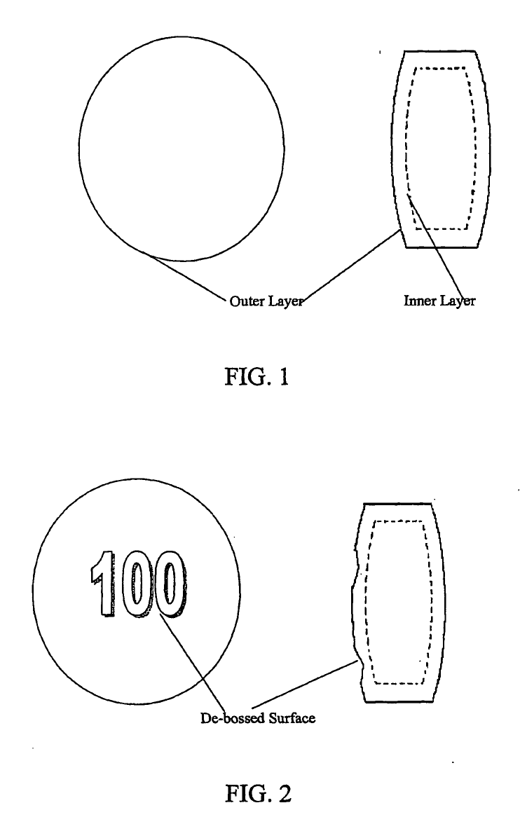

FIG. 1 illustrates a typical bi-convex tablet having an inner layer and an outer layer.

FIG. 2 illustrates a typical bi-convex tablet having de-bossed, engraved or indicia identification marks on an outer layer and an intact inner layer (a tablet according to the present invention).

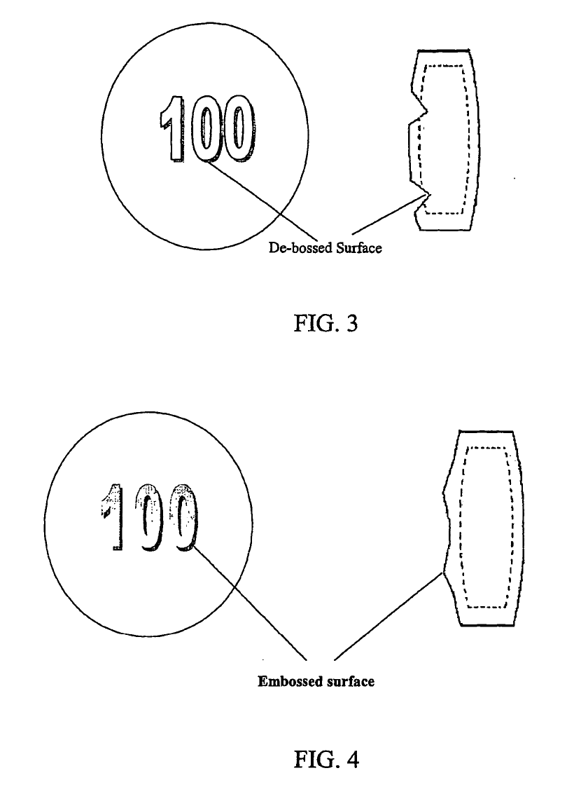

FIG. 3 illustrates a typical biconvex tablet having de-bossed, engraved or indicia identification marks on an outer layer and an non-intact inner layer.

FIG. 4 illustrates a typical bi-convex tablet having embossed identification marks on an outer layer and an inner layer.

### DETAILED DESCRIPTION OF THE INVENTION

The invention relates to a pharmaceutical composition for oral administration, comprising a core of active ingredient and an outer non-active layer or layers formed on the core by application of pressure. In a preferred embodiment the invention relates to a pharmaceutical composition containing 3-[2-(dimethylamino) ethyl]-indole-5-methanesulphonamide succinate (1:1), commonly known as Sumatriptan succinate, as active ingredient.

Sumatriptan and its physiologically acceptable salts and solvates are disclosed in UTK Patent Specification No. 2162522. Sumatriptan succinate exhibits selective vasoconstrictor activity and is useful in the treatment of migraine.

The invention seeks to obviate the unpleasant taste associated with oral administration of Sumatriptan succinate and/or increase stability of the active ingredient Thus the outer layer, or compression coating can eliminate the unpleasant taste associated with the Sumatriptan succinate. Preferably, compression coated tablet embodying the invention comprises a core containing an effective amount of 3-[2(dimethylamino)ethyl]-N-methyl-1H-indole-5-methanesulphonamide and salts thereof as active ingredient and optionally inactive ingredients, and a compression coat of pharmaceutical carriers or excipient over the core.

The present invention therefore provides a particularly advantageous compression coated solid dosage form suitable for oral administration of Sumatriptan succinate.

It will be understood that a pressure or compression-coated solid dosage form of a pharmaceutical composition means a solid core comprising the active ingredient and optionally pharmaceutical carriers or excipients, which is substantially covered with a compression coating of pharmaceutical carriers or excipients.

It has been found that the unpleasant taste associated with oral administration of the Sumatriptan succinate is substantially eliminated by the formulations of the present invention. It is important to note that these advantages are attained without any significant change in the dissolution profiling of sumatriptan succinate when compared to prior coated tablet formulations for oral administration.

It is preferred that 3-[2-dimethylamino)ethyl]-N-methyl-1H-indole-5-methanesulphonamide should be employed in compositions embodying the invention in the form of a physiologically acceptable salt Most preferably 3-2-dimethylamino)ethyl]-N-methyl-1H-indole-5-methanesulphonamide will be employed in such compositions embodying the invention in the form of its succinate (1:1) salt.

The ratio of core to outer compressor applied layer or coat is in the range 0.1:1 and most preferably in the range of 0.3:1 to 0.7:1.

In addition to the sumatriptan succinate thereof compositions of the invention will preferably comprise pharmaceutically acceptable carriers and excipients, alone or in combination such as binding agents.

Examples of binders are: acacia mucilage 0 to 25% w/v, preferably 1 to 5% w/v, alginic acid 0 to 20.0% w/v, preferably 1 to 5% w/v, polyvylpyrrolidone (povidone) 0 to 15.0% w/v, preferably 0.5 to 5% w/v, gelatin 0 to 20.0% w/v, preferably 1 to 5.0% w/v, sucrose 0 to 70.0% w/v, preferably 2.0 to 20.0% w/v, starch mucilage 0 to 10.0% w/v, preferably 0.5 to 5.0% w/v, pregelatinised staxch 0 to 10.0% wtv, preferably 0.5 to 5.0% w/v, starch paste 0 to 10.0% w/v, preferably 5.0 to 10.0% w/v, sodium alginate 0 to 5.0% w/v, preferably 1.0 to 3.0% w/v, sorbitol 0 to 10.0% w/v, preferably 3.0 to 10.0% w/v, tragacanth 0 to 20% w/v, preferably 5.0 to 10.0% w/v, glucose 0 to 50%, preferably 5 to 25% w/v, hydroxypropylmethyl cellulose (EIPMC) 0 to 10% w/v, preferably 1.0 to 5.0% w/v, magnesium aluminium silicate 0 to 40% w/v, preferably 2 to 10% w/v, starch paste 0 to 25% w/v, preferably 5 to 15% w/v, polyvinylpnrolidone 0 to 15% w/v, preferably 3 to 10% w/v, carboxymethylcellulose sodium 0 to 10% w/v, preferably 1 to 6% w/v, dextrin 0 to 50% w/v, preferably 5 to 25% w/v, ethyl cellulose 0 to 10% w/v, preferably 1 to 6% w/v, polyethylene glycol 0 to 5% w/v, guar gum 0 to 10% w/v, preferably. 1 to 5% w/v, zein 0 to 30% w/v, preferably 1 to 10% w/v, hydroxyethyl cellulose 0 to 5% w/v, preferably 2 to 4% w/v, hydroxypropyl cellulose up to 5% w/v, preferably 2 to 4% w/v, methyl cellulose up to 20% w/v, preferably 1 to 10% w/v, polymethacrylates up to 25% w/v, preferably 5 to 10% w/v, carboxymethylcellulose calcium 0 to 20% w/v, preferably 5 to 10% w/v.

b) Disintegrating agents: Tablets embodying the invention can be formulated in the absence of disintegrating agents although their inclusion may be advantageous for their disintegration in water. Examples of suitable disintegrating agents which can optionally be incorporated into a tablet according to the invention are: croscarnellose sodium 0 to 10% w/w, microcrystalline cellulose (e.g. Avicel R) 0 to 30% w/w, preferably 5 to 10% w/w, Sodium carboxymethyl cellulose (e.g. Nymcel R) 0 to 5% w/w, preferably 1 to 2% w/w, calcium carboxymethyl cellulose 0 to 20% w/w, preferably 1 to 5% w/w, modified cellulose gum (e.g. Ac-Di-Sol R) 0 to 10% w/v, preferably 1 to 5% w/w, cross-linked povidone 0 to 10% w/w, preferably 2 to 6% w/w, alginic acid and alginates 0 to 10% w/w, 2 to 5% w/w, pregelatinised starch 0 to 10% w/w, preferably 0.5 to 5% w/w, sodium starch glycollate (e.g. Explotab R, Primojel R) 0 to 10% w/w, preferably 0.5 to 5% w/w, modified corn starch (e.g. starch 1500 R) 0 to 20% w/w, preferably 1 to 10% w/w, starch (e.g. potatohinaize starch) 0 to 15% w/w, preferably 0.2 to 10% w/w, ion exchange ressn such as polacrin potassium (e.g. Amberlite IRP-88) up to 5% w/w, preferably 0.5 to 2.0% w/w.

c) Fillers: These serve the purpose of bulking up the tablet to a suitable size and aiding compressibility especially in lower dosage tablets. The amount of filler depends on its type, size of tablet and amount of active compound. When the concentraiion of active compound is below 60% w/w, more preferably 45% w/w and most preferably below 30% w/w, is advantageously used. Examples of water-soluble fillers (which can be used in general quantities of 0 to 95% w/w) are: soluble lactose, compressible sugar, confectioners sugar, dextrose, marmitol, sodium chloride, sorbitol xylitol sodium chloride F. Examples of water-insoluble fillers (which can be used in general quantities of 0 to 93% w/w) are: calcium carbonate, magnesium carbonate, calcium phosphate (e.g. di and tri basic calcium phosphate), calcium sulphate, kaolin, microcrystalline cellulose, powdered cellulose, pregelatinized starch 5 to 75%, starch, barium sulphate, magnesium trisilicate, aluminium hydroxide.

d) Lubricants: Generally lubricants are used in as low an amount as possible. Examples of lubricants with percentage weights which are suitable for a tablet are: stearates (e.g. magnesium or calcium stearate) 0.2 to 5% w/w, preferably 0.25 to 1% w/w, talc 0.19 to 5% w/w, preferably 1 to 2% w/w, polyethylene glycol 0.19 to 5% w/w, preferably 2 to 5% w/w, liquid paraffin 0.18 to 5% w/w, preferably 2 to 5% w/w, sodium lauryl sulphate 0.19 to 5% w/w, preferably 0.5 to 2% w/w, magnesium lauryl sulphate 0.12 to 5% w/w, preferably 1 to 2% w/w, colloidal silicon dioxide 0.1 to 5% w/w, preferably 0.1 to 1.0% w/w, palmitostearate 0.01 to 5% w/w, preferably 1 to 3% w/w, stearic acid 0.01 to 5% w/w, preferably 1 to 3% w/w, zinc stearate 0.01 to 2% w/w, 0.5 to 1.5% w/w, hydrogenated vegetable oil 0.5 to 5% w/w, preferably 1 to 3% w/w. More suitably the lower value is 0.25%.

e) Wetting agents/surfactants: examples with suitable amounts are: sodium dodecyl sulphate 0 to 10% w/w, preferably 0.5 to 2% w/w, sodium lauryl sulphate 0 to 10% w/w, preferably 0.1 to 3.0% w/w, polyoxyethylene sorbitan fatty acid esters Cweens) 0 to 3% w/w, preferably 0.05 to 1.0% w/w, polyoxyethylene stearates 0 to 2% w/w, preferably 0.05 to 1.0% w/w, sorbitan fatty acid esters (Spans) 0 to 3% w/w, preferably 0.05 to 1.0% w/w.

f) Glidants: for example, talc 0 to 5% wIw, preferably 1 to 2% w/v, starch 0 to 15% w/w, preferably 2 to 10% w/w, magnesium stearate up to 5%, preferably 0-2.0% w/w, silica derivatives generally 0 to 1% w/w, preferably 0.2 to 0.5% w/w, such as colloidal silica (e.g. Aerosil) 0 to 0-5% w/w, preferably 0.25 to 3% w/w, pyrogenic silica 0 to 2% w/w, preferably 0.25 to 1% w/w, hydrated sodium silicoaluminate 0 to 2% w/w, preferably 0.5 to 1% w/w, colloidal silicon dioxide 0 to 0.5% w/w.

g) Flavouing agents and flavour enhancing agents are used alone or in combination, for example Ethyl Maltol, Ethyl vanillin, Fuaric acid, Malic acid, Tartric acid, Maltol, Menthol Vanmin, fruity flavours and combinations thereof, approximate quantities being 0 to 5% w/w, preferably 0.25 to 2% w/w, of fruity flavours.

h) Sweetening agents: for example sodium saccharin 0 to 10% w/w, preferably, 0.5 to 5.0% w/w, asparrame 0 to 10% w/w, preferably 0.25 to 5.0% w/w, confectioners sugar 0 to 30% w/w, preferably 5 to 20% w/w, sorbitol 25 to 90% w/v, preferably 0.5 to 10% w/w, sucrose 0 to 85% w/w, preferably 0.5 to 20% w/w, xylitol 020% w/w, preferably 0.5 to 10% w/w, glycyrduzinic acid and its derivatives 0.2 to 2.0% w/w.

Such materials may be incorporated at the appropriate stage(s) of the manufacturig process together with any other agents (e.g colorants).

For the preparation of compositions embodying the invention, sumatriptan succiate is optionally blended with suitable excipients and granulated. Preferably sumatriptan succinate will be granulated with filler before compression coating. Most preferably the filler employed will be lactose. The filler may be granulated separately with the binder solution. Preferred solvents for granulation are water and alcohols. The solvent does not appear in the final product The amount of solvent may be varied according to the total weight of solid dosage form. In the present invention, therapeutic agent alone or optionally with pharmaceutical-carriers or excipients is pre-compressed to make a solid core which is then compressed coated with pharmaceutical carriers or excipients by using a press coat machine.

The amount of sumaptriplan, preferably in the form of a physiologically acceptable salt, employed in the compositions of the invention wim preferably be in the range of about 25 mg to about 200 mg, most preferably about 25 mg to 100 mg, expressed as the weight of free base.

A preferred aspect of the invention is to thus eliminate the unpleasant taste associated with oral administration of Sumatriptan succinate. The compression coating eliminates the unpleasant taste associated with the Sumatriptan succinate. Compression coated tablets are therefore provided comprising a core containing an effective amount of Sumatriptan succinate thereof as active ingredient and optionally inactive ingredients, and a compression coat of inactive ingredients over the core.

We have found the most advantageous composition of the core to) include a disintogrant of about 2.50 to 10.0 mg croscannellose sodium, a filler of about 10 to 150 mg lactose, and a lubricant of about 0.87 to 3.50 mg magnesium stearate. The amounts of each ingredient vary depending on the amount of sumatriptan in the tablet

The most advantageous corrposition of the outer layer is about 160 to 300 mg lactose, about 35 to 60 mg microcrystalline celblwose, about 7.0 to 12.0 mg crocarelose sodim, and about 2.00 to 3.50 mg mnagnesium stearte. The amounts of each ingriet vary depending on the amount of sumatriptein in the core tablet.

The invention is flrter illste by the following non-limiting examples wherein the active ingredient is sumatriptan succinate (1:1).

### EXAMEPLES

### Example 1

| | |
|---|---|
| Unit formula | (mg/tablet) |

Core:

| | |
|---|---|
| Active ingredient ** | 140.0 |
| Excipients (optional) | 70.0 |
| Lubricant | q.s. |

Compression coat (Wet granulation process):

| | |
|---|---|
| Lactose (Ph Eur.) | 266.0 |
| Microcrystalline Cellulose (Ph Eur) | 59.0 |
| Croscarmellose Sodium (USP NF) | 12.0 |
| Magnesium Stearate (Ph Eur) | 3.5 |
| Purified water (Ph Eur) | q.s.+ |

+The water does not appear in the final product.
** Equivalent to 100 mg free base

The active ingredient alone or with one or more suitable excipients was granulated with water. Other granulating agents such as polyvinyl pyrrolidone may be used but was not essential for the present work. The granules obtained were dried and passed through a screen, and the resulting granules were then mixed with the other tablet core excipients. The mix was pre-compressed to make a solid core. The solid core containing active ingredient was compression coated using coating granules. Coat granules, which do not contain any active ingredient, were made by a standard wet granulation process.

### Example 2

| | |
|---|---|
| Unit formula | (mg/tablet) |

Core:

| | |
|---|---|
| Active ingredient ** | 140.0 |
| Lactose (Ph. Eur.) | 60.0 |
| Purified water Ph Eur | q.s.+ |

Compression coat (Wet granulation process):

| | |
|---|---|
| Lactose (Ph. Eur.) | 266.0 |
| Microcrystalline Cellulose (Ph. Eur.) | 59.0 |
| Croscarmellose Sodium (USP NF) | 12.0 |
| Magnesium Stearate (Ph Eur) | 4.0 |
| Purified water Ph Eur | q.s.+ |

+The water does not appear in the final product.
** Equivalent to 100 mg free base

The active ingredient and lactose were granulated with water. The granules obtained were dried and passed through a screen, and the resulting granules were then mixed with the other tablet core excipients like disintegrating agent and lubricating agent The mix was pre-compressed to make a solid core. The solid core containing active ingredient was compression coated using coating granules. Coat granules, which do not contain any active ingredient, were made by a standard wet granulation process.

### Example 3

| | |
|---|---|
| Unit formula | (mg/tablet) |

Core:

| | |
|---|---|
| Active ingredient ** | 140.0 |
| Excipients (optional) | 70.0 |
| Lubricant | q.s. |
| Purified water (Ph Eur) | q.s.+ |

Compression coat (Direct Compression Process):

| | |
|---|---|
| Lactose anhydrous (Ph Eur) | 266.0 |
| Microcrystalline Cellulose (Ph Eur) | 59.0 |
| Croscarmellose Sodium (USP NF) | 12.0 |
| Magnesium Stearate (Ph Eur) | 3.5 |
| Purified water (Ph Eur) | q.s.+ |

+The water does not appear in the final product.
** Equivalent to 100 mg free base

The active ingredient alone or with one or more suitable excipients was granulated with water. The granules obtained were dried and passed through a screen, and the resulting granules were then mixed with the other tablet core excipients. The mix was pre-compressed to make a solid core. The solid core containing active ingredient was compression coated by the process of direct compression using the above directly compressible powder blend Coating powder blend, not containing any active ingredient, was made by a standard mixing process.

Sumatriptan surcinate tablets were produced using stdd biconcave tooling without any marking on tablet surface using formula as for Example No 4.

### Example 4

| | |
|---|---|
| Unit formula | (mg/tablet) |

Core:

| | |
|---|---|
| Active ingredient ** | 140.0 |
| Lactose Monohydrate (Intragranular) | 57.50 |
| Crosscarmellose Sodium (Intragranular) | 5.0 |
| Purified Water | q.s.+ |
| Crosscarmellose Sodium (Extragranular) | 5.0 |
| Magnesium Stearate (Extragranular) | 3.50 |

Compression coat:

| | |
|---|---|
| Lactose Monohydrate (Intragranular) | 266.0 |
| Microcrystalline Cellulose (Intragranular) | 58.50 |
| Croscarmellose Sodium (Intragranular) | 6.00 |
| Purified Water | q.s.+ |
| Crosscarmellose Sodium (Extragranular) | 6.00 |
| Magnesium Stearate (Extragranular) | 3.50 |

+The water does not appear in the final product.
** Equivalent to 100 mg free base

The active ingredient alone or with one or more suitable excipients was granulated with the Purified Water. The granules obtained were dried and passed through the screen, and the resulting granules were then mixed with the other tablet core excipients. The mix was pre-compressed to make a solid core.

Lactose Monohydrate, Microcrystalline Cellulose and Crosscarmellose Sodium were mixed together and granulated with Purified Water. The granules obtained were dried and passed through the screen, and the resulting granules were then mixed with the Crosscarmellose Sodium and Magnesium Stearate.

The solid core containing active ingredient and outer layer of inactive diluents were compressed.

Additional experiments were performed using standard biconcave tooling with identification logo, code numbers, and strength de-bossed on the tablet surface as for Example No 4. It was expected that de-bossing of the outer tablet layer would produce cracks and cause the inner layer (with bitter active agent) to be exposed to the atmosphere. This would result in the tablet having a bitter taste; or if the active agent was degraded by the atmosphere, in the active agent losing effectiveness.

Surprisingly the debossing of the tablet surface did not affect the taste and release profile of the product when evaluated in the laboratory. Technical challenge of providing a cost effective means of tablet identification for bilayer or multilayer tablets without associated release of; or exposure of, the inner layer(s) was achieved. Tablets of other strengths with different debossing were also developed.

An additional technological feature associated with present invention is that the inner and outer layer does not include an external binding agent

In all embodiments, it will be understood that the core layer of a composition embodying the invention will be gradually removed by a combination of dissolution and erosion or rapid disintegration once exposed to a particular environment, and after gradual removal of the outer layer, which occurs on administration.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. It is intended, therefore, that the invention be defined by the scope of the claims which follow and that such claims be interpreted as broadly as is reasonable.

What we claim is:

1. A taste masking pharmaceutical composition for oral administration, comprising a core of active ingredient and one or more outer non-active taste masking layers formed on the core by application of pressure.

2. A composition according to claim 1, in which there is a plurality of superposed non-active layers.

3. A composition according to claim 2, wherein each layer from the layer adjacent the core, to the outermost layer is formed in turn by application of pressure.

4. A composition according to claim 1, wherein each layer forming the outer layer, is applied by a compression method to form the outer layer.

5. A composition according to claim 4, wherein the outer non-active layer comprises powder blends or granules which are compressed onto the core to form said outer layer.

6. A composition according to claim 5, wherein the powder blends or granules are formed by either a wet granulation process, or a dry compaction or slugging de-slugging process, or a direct compression process.

7. A composition according to claim 1, the outer layer encases substantially the whole surface area of the core.

8. A composition according claim 1, wherein the outer surface of the outer layer comprises a surface profile.

9. A composition according to claim 8, wherein the surface profile is an engraved surface profile.

10. A composition according to claim 8, wherein the surface profile is an intaglio surface profile.

11. A composition according to claims 8, further comprising an applied indicia.

12. A composition according to claim 11, the applied indicia comprising a printed indicia.

13. A composition according to claim 1 wherein the core further comprises one or more pharmaceutical excipients.

14. A composition according to claim 1, wherein a outer non-active layer comprises one or more pharmaceutical carrier(s) and excipient(s).

15. The composition according to claim 1, wherein an outer layer comprises a lubricating agent, a disintegrating agent, and a diluting agent.

16. A composition according to claim 15, wherein the lubricating agent is about 0.1 to 5% magnesium stearate, the disintegrating agent is about 0.05 to 15% croscarmellose sodium, and the diluting agent is about 30 to 90% microcrystalline cellulose.

17. A composition according to claim 1, in which the outer layer is further film coated.

18. A composition according to claims 1, wherein the outer layer is formed by a multiple compression method.

19. A composition according to claim 1, wherein the core comprises 0.01 mg to 1000 mg of one or more active ingredients.

20. A composition according to claim 1, wherein the core comprises more than one active ingredients.

21. A composition according to claim 1, wherein the active ingredient or ingredients are selected from the group consisting of sumatriptan and its pharmacologically acceptable salts.

22. A composition according to claim 21, the active ingredient comprising sumatriptan succinate.

23. A taste masking pharmaceutical composition for oral administration, comprising a core of sumatriptan and one or more outer non-active layers formed on the core by application of pressure.

24. The composition of claim 23 wherein the sumatriptan is provided as a pharmaceutically acceptable salt.

25. The composition of claim 24 wherein the samaritan is the succinate salt.

26. The composition of claim 23 wherein the core contains about 25 to 200 mg of sumatriptan calculated as the free base.

27. The composition of claim 23 wherein the core contains about 25 to 100 mg of sumatriptan calculated as the free base.

28. The composition of claim 23 wherein the core comprises sumatriptan or a pharmaceutically acceptable salt thereof and one or more pharmaceutically acceptable excipients selected from the group comprising binders, disintegrating agents, fillers, lubricants, wetting agents, surfactants, glidants, flavoring agents, sweetening agents, and colorants.

29. The composition of claim 23 wherein the sumatriptan or a pharmaceutically acceptable salt thereof is granulated with a disintegrate, a filler, and a lubricant, and compressed to make the core.

30. The core of claim 29, wherein the disintegrate is about 2.50 to 10.0 mg croscarmellose sodium, the filler is about 10 to 150 mg lactose, and the lubricant is about 0.87 to 3.50 mg magnesium stearate.

31. The composition of claim 23 wherein the non-active layers comprise one or more pharmaceutically acceptable excipients selected from the group comprising binders, disintegrating agents, fillers, lubricants, wetting agents, surfactants, glidants, flavoring agents, sweetening agents and colorants.

32. The composition of claim 23, wherein the non-active outer layer comprises a filler, one or more disintegrating agents, and a lubricant.

33. The outer layer of claim 32, comprising about 160 to 300 mg lactose, about 35 to 60 mg microcrystalline cellulose, about 7.0 to 12.0 mg croscarmellose sodium, and about 2.00 to 3.50 mg magnesium stearate.

34. A method of treating migraine which comprises administering the composition of claim 23.

35. A process for preparing taste masked pharmaceutical tablet for oral administration comprising the steps of:
forming core of active ingredient; and
applying at least one taste-masking layer of pharmaceutical excipients on the active core by compression.

36. A dosage form of sumatriptan comprising inner core of sumatriptan with one or more pharmaceutically acceptable excipients and at least one outer compressed layer of pharmaceutically acceptable excipients.

37. A method treating a human suffering from migraine which comprises administering the composition of claim 36.

38. A process for preparing sumatriptan tablet for oral administration comprising the steps of:
forming core of sumatriptan or pharmaceutically acceptable salt thereof, and
applying at least one layer of pharmaceutical excipients on the core by compression.
